(19) United States
(12) Patent Application Publication
(10) Pub. No.: US 2016/0303301 A1
(43) Pub. Date: Oct. 20, 2016

(54) IMPLANTABLE POWER PACK

(71) Applicant: MINNETRONIX, INC., Saint Paul, MN (US)

(72) Inventors: Vlad Bluvshtein, Plymouth, MN (US); Lori Lucke, Rosemount, MN (US)

(21) Appl. No.: 15/096,886

(22) Filed: Apr. 12, 2016

Related U.S. Application Data
(60) Provisional application No. 62/147,383, filed on Apr. 14, 2015.

(51) Int. Cl. A61M 1/10; A61M 1/12; H02J 7/04; H02J 50/80; H02J 50/12; H02J 7/02

(52) U.S. Cl. CPC A61M 1/1086; H02J 50/12; H02J 7/025; H02J 7/045; H02J 50/80; A61M 1/122; H02J 7/345

(57) ABSTRACT

Disclosed is an implantable power pack. In one implementation, the implantable power pack includes a housing, a power output port disposed on the housing, a resonant network configured to receive a wireless power transfer from an external primary power source, a power transfer secondary electronics component configured to convert alternating current power received from the resonant network to direct current power, and a power management component configured to provide the direct current power through the power output port.

### CROSS-REFERENCE TO RELATED APPLICATIONS

This application claims the benefit of U.S. Provisional Patent Application No. 62/147,383, filed Apr. 14, 2015 and entitled “IMPLANTABLE POWER PACK,” The entirety of which is herein incorporated by reference.

### TECHNICAL FIELD

The technology described herein relates to systems and methods for providing electric power to implanted medical devices through an implanted power pack.

### BACKGROUND

There is a need to deliver electric power to implanted medical devices such as artificial hearts and ventricle assist devices. It is possible to deliver power non-invasively through electromagnetic energy transmitted through the skin. An implantable system for a device such as left ventricle assist device (LVAD) controller includes at least internal and external controllers, power transfer coils, batteries, and a pump. The components that are implanted and therefore internal to the subject include the internal power transfer coil, the internal battery, the internal controller, and the pump. In a typical arrangement, the internal controller, the internal battery, and the pump are implanted in the same package. Thus, when the battery wears out and needs to be replaced, the entire package may need to be surgically removed despite the fact that some components such as the controller or pump are stilling functioning normally. Prior art systems fail to address these and other issues that concern transfer of electromagnetic energy to implanted medical devices. These and other deficiencies of the prior art are addressed herein.

### SUMMARY

In one aspect, the present disclosure is directed to an implantable power pack that includes a housing; a power output port disposed on the housing; a resonant network included within the housing and configured to receive a wireless power transfer from an external primary power source; a power transfer secondary electronics component included within the housing, connected to the resonant network, and configured to convert alternating current power received from the resonant network to direct current power; and a power management component included within the housing, connected to the power transfer secondary electronics component, and configured to provide the direct current power through the power output port.

In some implementations, the implantable power pack further includes a battery included within the housing and configured to be recharged by power transferred from the external primary power source.

In some implementations, the resonant network includes a coil and a capacitor.

In some implementations, the power transfer secondary electronics component includes a rectifier.

In some implementations, the power management component includes regulator.

In some implementations, the implantable power pack further includes a plurality power output ports disposed on the housing wherein the power management component provides direct current power through the plurality of power output ports.

In some implementations, the power management component provides electric power to a left ventricle assist device that is connected to the output port.

In some implementations, the housing is implanted within a subject in a location that is adjacent to the skin of the subject,

In some implementations, the power management component shifts between a power supply mode and an idle mode depending on power demanded from an implanted medical device that is connected to the output port.

In another aspect, the present disclosure is directed to an implantable system that includes a power pack configured to receive a wireless power transfer from an external primary power source and to provide power through a power output port disposed on a housing of the power pack; and a medical device configured to connect to the power output port of the power pack and to receive power from the power pack through a power input port disposed on a housing of the medical device, wherein the housing of medical device does not have a battery included therein.

In some implementations, the power pack includes a resonant network included within the housing and configured to receive a wireless power transfer from an external primary power source; a power transfer secondary electronics component included within the housing, connected to the resonant network, and configured to convert alternating current power received from the resonant network to direct current power; a power management component included within the housing, connected to the power transfer secondary electronics component, and configured to provide the direct current power through the power output port; and a battery included within the housing and configured to be recharged by power transferred from the external primary power source.

In some implementations, the medical device is a ventricle assist device including a pump that supports a heart function of a subject within whom the housing is implanted; a controller configured to control the operation of the pump; a sensor configured to detect pressure within a circulatory system of the subject and to provide the detected pressure as feedback to the controller; and an antenna that provides for wireless communication to external components; wherein the controller is configured to communicate status information and/or receive commands through the antenna.

In another aspect, the present disclosure is directed to an implantable medical device that includes a housing; a power output port disposed on the housing; a ventricle assist component included within the housing; a power pack component included within the housing and connected to the ventricle assist section and to the power output port, the power pack section configured to receive a wireless power transfer from an external primary power source and to provide power to the ventricle assist section and to an additional implanted medical device through the power output port.

In some implementations, the ventricle assist component includes a pump that supports a heart function of a subject within whom the housing is implanted; a controller configured to control the operation of the pump; a sensor configured to detect pressure within a circulatory system of the subject and to provide the detected pressure as feedback to the controller; and an antenna that provides for wireless communication to external components; wherein the controller is configured to communicate status information and/or receive commands through the antenna.

In some implementations, the power pack component includes a resonant network included within the housing and configured to receive a wireless power transfer from an external primary power source; a power transfer secondary electronics component included within the housing, connected to the resonant network, and configured to convert alternating current power received from the resonant network to direct current power; a power management component included within the housing, connected to the power transfer secondary electronics component, and configured to provide the direct current power through the power output port; and a battery included within the housing and configured to be recharged by power transferred from the external primary power source.

In another aspect, the present disclosure is directed to a ventricle assist device, that includes a housing; a power input port disposed on the housing; a pump that supports a heart function of a subject within whom the housing is implanted; and a controller configured to control the operation of the pump; wherein the pump and the controller receive electric power through the power input port and the housing does not have a battery included therein.

In some implementations, the implantable medical device further includes a sensor configured to detect pressure within a circulatory system of the subject and to provide the detected pressure as feedback to the controller.

In some implementations, the implantable medical device further includes an antenna that provides for wireless communication to external components; wherein the controller is configured to communicate status information and/or receive commands through the antenna,

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments of the invention and illustrated in the accompanying drawings.

### BRIEF DESCRIPTION OF THE DRAWINGS

FIG. 1 is a block diagram of a wireless power transfer system that includes an implanted power pack in accordance with embodiments discussed herein.

FIG. 2 is circuit diagram for certain components of the system shown in FIG. 1.

FIGS. 3A and 3B are schematic illustrations of the internal and external coils shown in FIG. 1.

FIG. 4 is a circuit diagram that shows one implementation of the inverter shown in FIG. 1.

FIG. 5 is an illustration of waveform traces for signals that are present in the system of FIG. 1 as power is transferred between the external assembly and the internal assembly.

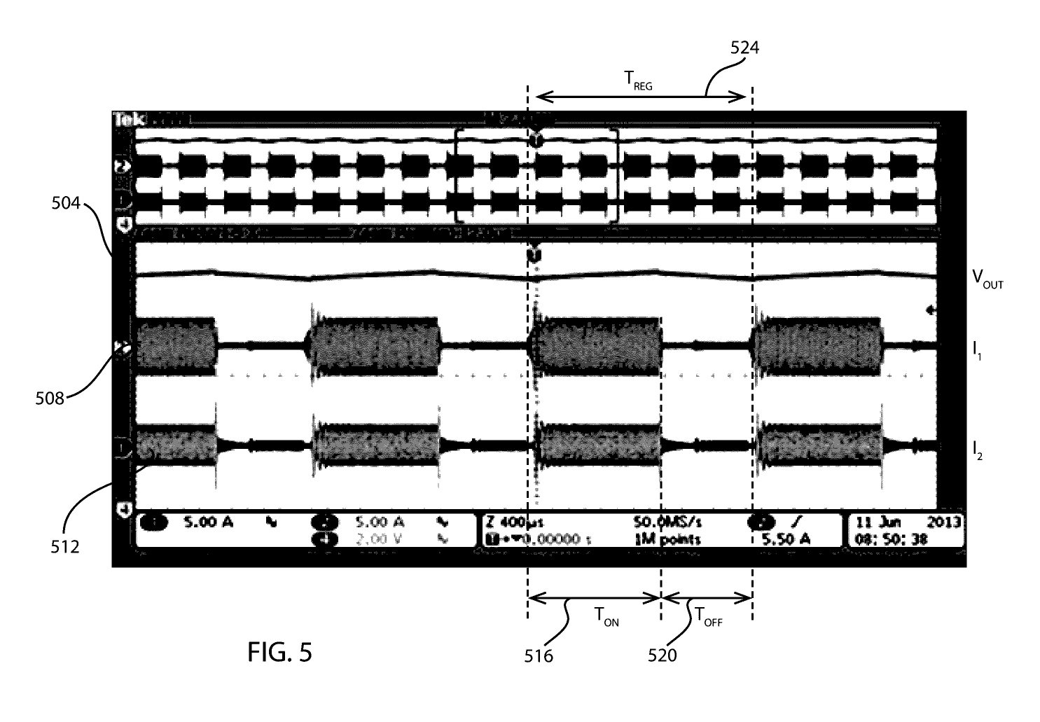

FIG. 6 is a schematic illustration of an example configuration where the implantable power pack of FIG. 1 provides power to a single implanted medical device.

FIG. 7 is a schematic illustration of an example configuration where the implantable power pack of FIG. 1 provides power to multiple implanted medical devices.

FIG. 8 is a schematic illustration of an example configuration that combines in one package the implantable power pack of FIG. 1 and a ventricle assist device,

### DETAILED DESCRIPTION

The present disclosure is directed to an implantable power pack for use in connection with a transcutaneous energy transfer system (TETS). The implantable power pack may be configured to be implanted within a subject and to provide electrical power to one or more medical devices, such as an artificial heart or ventricle assist device. Implantable power pack embodiments combine in one package a secondary power transfer coil, TETS electronics, a power management component, and a battery. The secondary power transfer coil and the TETS electronics are configured to receive a wireless power transfer from an external primary that is located outside of the subject. The power management component is configured to receive electrical power from the TETS electronics and to provide power to the one or more implanted medical devices through one or more power outputs associated with the implantable power pack.

### System Overview

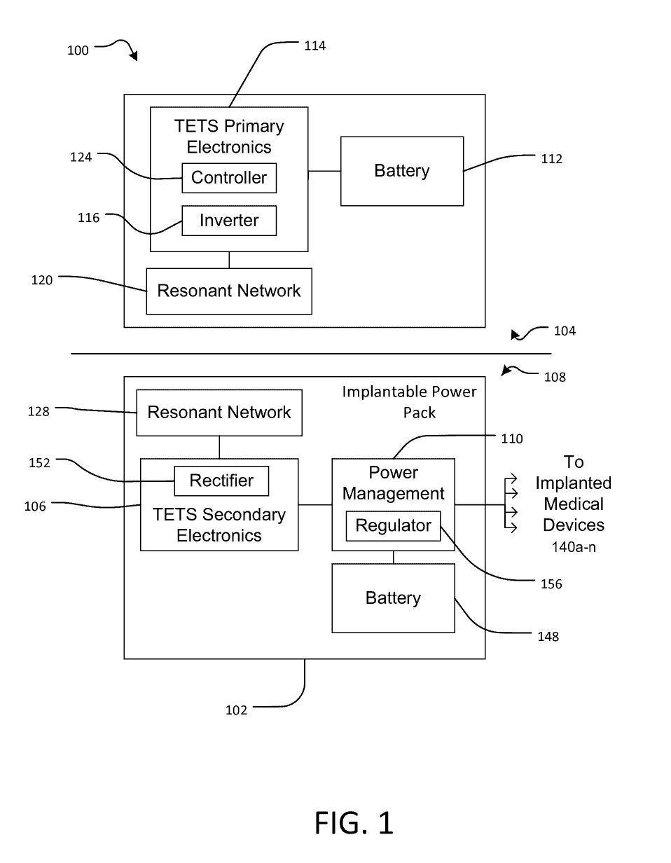

FIG. 1 is a schematic illustration of an implantable power pack 102 in accordance with embodiments discussed herein. As shown in FIG. 1, the implantable power pack 102 may operate as part of a wireless power transfer system 100. The system 100 may be referred to as a transcutaneous energy transfer system (TETS) when applied to implantable electronic applications. The system 100 has an external assembly 104 that is provided at an external location outside of a subject and an internal assembly 108 that is implanted within the subject. The internal assembly 108 includes the implantable power pack 102, as well as one or more implantable medical devices 140a-n. An implantable medical device used with the power pack 102 may be any medical device capable of being implanted in a subject, such as a heart pump, an artificial heart, a right ventricle assist device, a left ventricle assist device, a BIVAD, a minimally invasive circulatory support system, a cardiac pace maker, and so on. While the implanted device may be any implantable medical device, this disclosure describes the implantable power pack 102 in the context of a heart pump by way of example and not limitation.

An implantable power pack 102 may include a coil or other internal resonant network 128 that is configured to receive a wireless power transfer from the external assembly 104. The internal resonant network 128 is provided in association with TETS secondary electronics 106. The TETS electronics 106 may include at least a rectifier 152 that converts alternating current (AC) power received from the external assembly 108 to direct current (DC) power. The TETS secondary electronics 106 is operably connected to a power management component 110, which is generally configured to manage aspects of power transfer including distributing direct current power from the TETS electronics 106 to the one or more medical devices 140a-n connected to implantable power pack 102. The implantable power pack 102 may additionally include a battery 148, which in some implementations is replaceable. Power transferred from the external primary 104 may additionally be used to recharge the battery 148 which, in turn, may provide an alternate source of power when power is not available from the external primary 104.

A power pack 102 in accordance with the present disclosure may be configured to be implanted in a location that is generally adjacent to the skin of the subject rather than deeper within the body of the subject. Implanting in this location positions the power pack 120 for an efficient power transfer coupling with the external primary. Additionally, in this location, the power pack 102 may be more surgically accessible in the event that the power pack 120 or components of the power pack 102 need to be repaired or replaced. For example, the battery 148 associated with power pack 102 may have a relatively shorter life span than other components and so may need to be periodically removed and replaced. Because the power pack 102 is located near the surface of the subject's body, the power pack 102 may be accessed and the battery 148 replaced though a less invasive surgical procedure than would be necessary if the battery 102 were located deeper within the patient's body.

### Wireless Power Transfer to the Implemented Power Pack

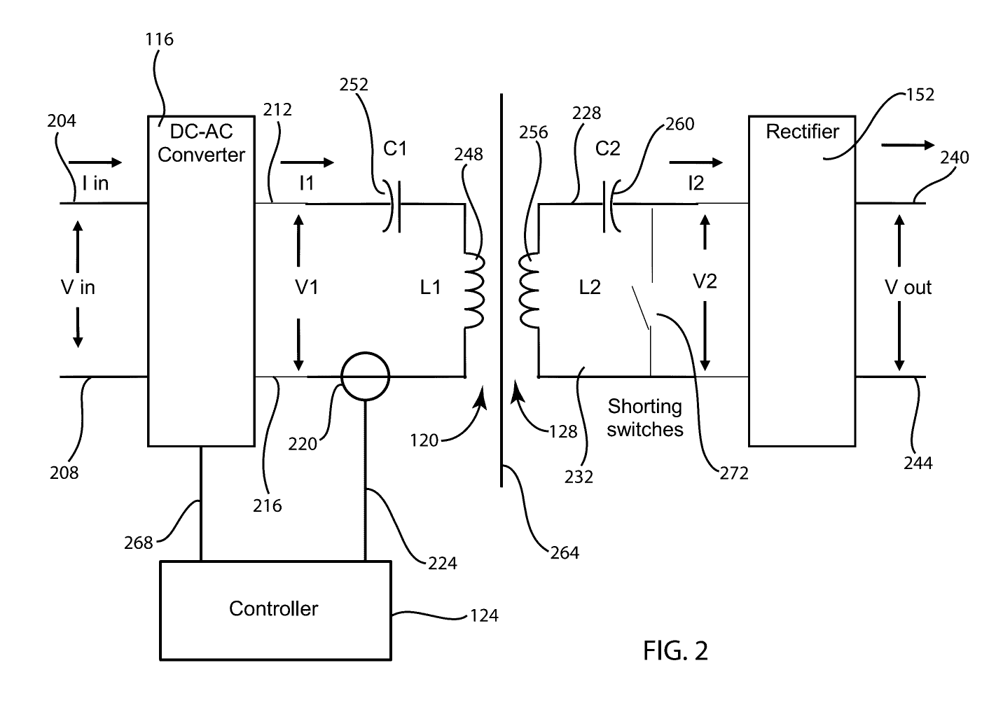

As shown in FIG. 1, the implantable power pack 102 may include an internal resonant network 128. Similarly, the external assembly 104 may include an external resonant network 120. The internal resonant network 128 and the external resonant network 120 are also shown in FIG. 2, which is a circuit diagram that includes certain components of the transcutaneous energy transfer system 100. As shown in FIG. 2, the external resonant network 120 may include an external coupler in the form of an inductive coil 248 and a capacitor 252 connected in series. Similarly, the internal resonant network 128 may include an internal coupler in the form of an inductive coil 256 and a capacitor 260 connected in series. It should be appreciated that the series-series topology illustrated in FIG. 2 is shown by way of example and not limitation. Alternative embodiments may be used that employ different circuit topologies, such as series-parallel, parallel-series, parallel-parallel and so on.

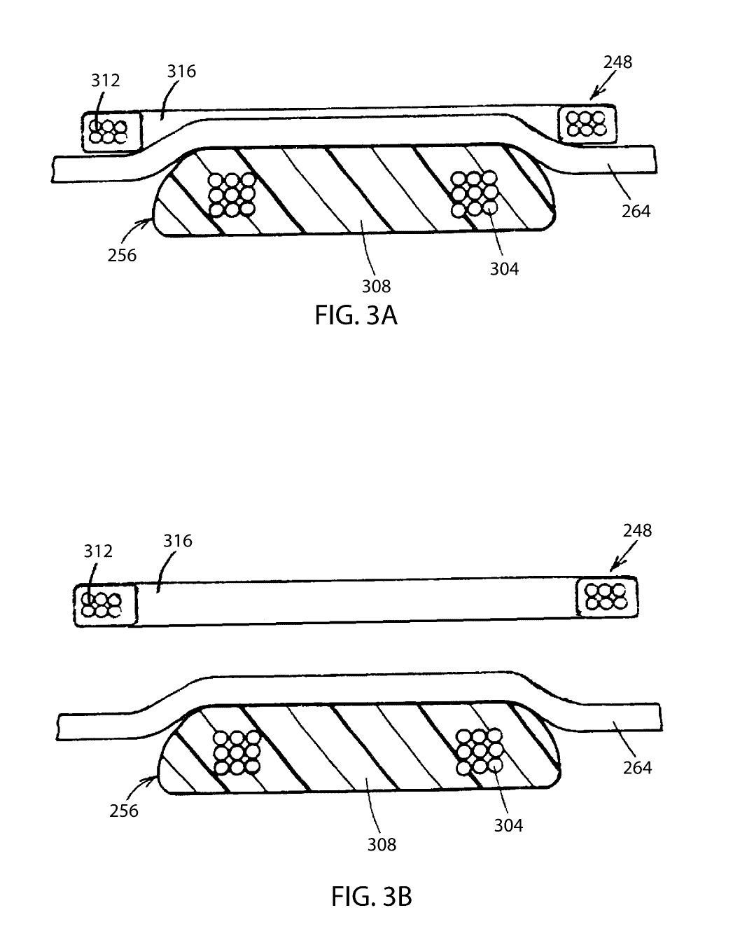

FIGS. 3A and 3B are schematic illustrations of the internal 256 and external 248 coils. As mentioned, an implantable power pack 102 may be implanted generally adjacent to the skin of the subject so as to provide for an efficient power transfer coupling with the external primary 104, among other advantages. Thus, as shown in FIG. 3A, the internal coil 256 may be disposed beneath the skin 264 of a subject. The external coil 248 may be disposed generally adjacent the internal coil 256. The external coil 248 may move with respect to the subject. Thus, as shown in FIG. 3B, the internal coil 256 is disposed beneath the skin 264 of a subject, and the external coil 248 is disposed at some distance from the internal coil 256. As shown in FIGS. 3A and 3B, the internal coil 256 may have a plurality of conductive windings 304 disposed in a circular insulating member 308. Similarly, the external coil 248 may have a plurality of conductive windings 312 disposed in an insulating ring 316. The inductance of each of the coils 256, 248 may be determined by the number, diameter and spacing of the windings 304, 312. The inductive or electromagnetic coupling between the coils 248, 256 is a function of their physical proximity, operating frequencies, coil sizes, and inductances. While the coils shown in FIGS. 3A and 3B have a generally circular shape, other shapes and structures may be used to implement the internal 256 and external 248 coils, depending on the implementation. For example, the coils 248, 256 may be shaped as a triangle, square, rectangle, pentagon, octagon, and so on. Generally, the coils 248, 256 may be shaped as polygons of any number of sides, which may be equal or unequal in length. The coils may be straight in certain portions and/or curved in certain portions. The coils 248, 256 may be arranged in a planar configuration. Alternatively, the coils 248, 256 may be arranged such that portions of the coils lie in different planes,

The coils 248, 256 together constitute a loosely coupled transformer, with the external coil 248 acting as a primary winding and the internal coil 256 acting as a secondary winding. The coils 248, 256 and the capacitors 252, 260 with which they may be connected may form a resonant circuit. The coils 248, 256 may be tuned to the same or different resonant frequencies. For example, the coils 248, 256 may be series tuned to a power transmission frequency of about 200 kHz. The external coil 248 may induce an electric current in the internal coil 256, which current generally behaves in accordance with the following equation:

$$\frac{{V}_{1}}{{I}_{2}} = \frac{{V}_{2}}{{I}_{1}} = ω \cdot k \cdot \sqrt{{L}_{1} \cdot {L}_{2}} \qquad ( 1 )$$

In Equation (1), I₁ is the current induced in the external resonant network 120. I₂ is the current induced in the internal coil network 128, is the voltage across the external resonant network 120, V₂ is the voltage across the internal resonant network 128. ω is the frequency of the voltage across the coils 248, 256, where the coil networks are tuned to the same frequency ω. L₁ is the inductance of the external coil 248. L₂ is the inductance of the internal coil 256. k is the coupling coefficient.

Referring to both FIG. 1 and FIG. 2, the external assembly 104 may include TETS primary electronics 114 and a power supply 112. Generally, the power supply 112 provides power in the form of a DC voltage and TETS primary electronics 114 converts or otherwise delivers the power for transfer through the external resonant network 120. In some embodiments, the power supply 112 is a portable battery or battery pack providing a DC voltage of between 10 and 18 volts. The TETS primary electronics 114 may include an inverter 116 connected to the power supply 112 via a pair of conductive lines 204, 208. The power supply 112 supplies the DC voltage to the inverter 116, which converts the DC voltage into a high-frequency voltage. The high-frequency voltage is provided to the external resonant network 120 via a pair of conductors 212, 216. A current sensor 220 may be used to sense the electric current flowing within the conductor 216. The current sensor 220 may be configured to sense either or both of the magnitude and phase of the electric current in the conductor 216. A controller 124 connected to the current sensor 220 via a conductor 224 may be used to control the operation of the inverter 116, based on one or more characteristics of the current sensed by the sensor 220. The controller 124 may also be configured to control the voltage Vᵢₙ that is provided by the power supply 112. The external coil network 120, which is disposed adjacent the skin 264 of the subject, transfers electric power through the skin 264 of the subject to the internal coil network 128 disposed beneath the skin 264 of the subject.

The implantable power pack 102 is disposed beneath the skin 264 of the subject and includes the internal coil network 128. As mentioned, the internal coil network 128 may be connected to a TETS secondary electronics component 106. The TETS secondary electronics component 106 may include a rectifier 152 that performs full wave rectification of the sinusoidal AC current induced in the internal coil 256 by the external coil 248. As shown in FIG. 2, the internal resonant network 128 and the rectifier 152 may be connected via a pair of conductors 228, 232. In one embodiment, the rectifier 152 includes four switching elements, which may be provided in the form of diodes or Schottky diodes. During a first half of the AC power cycle, a first pair of diodes provides a conductive path up from ground, through the internal coil 256, and out to conductor line 228. During a second half of the AC power cycle, a second pair of diodes provides a conductive path up from ground, through the internal coil 256, and out to conductor line 228. In this way, the rectifier 152 converts AC power provided by the internal coil network 128 into DC power that can be used by various components of the internal assembly 108.

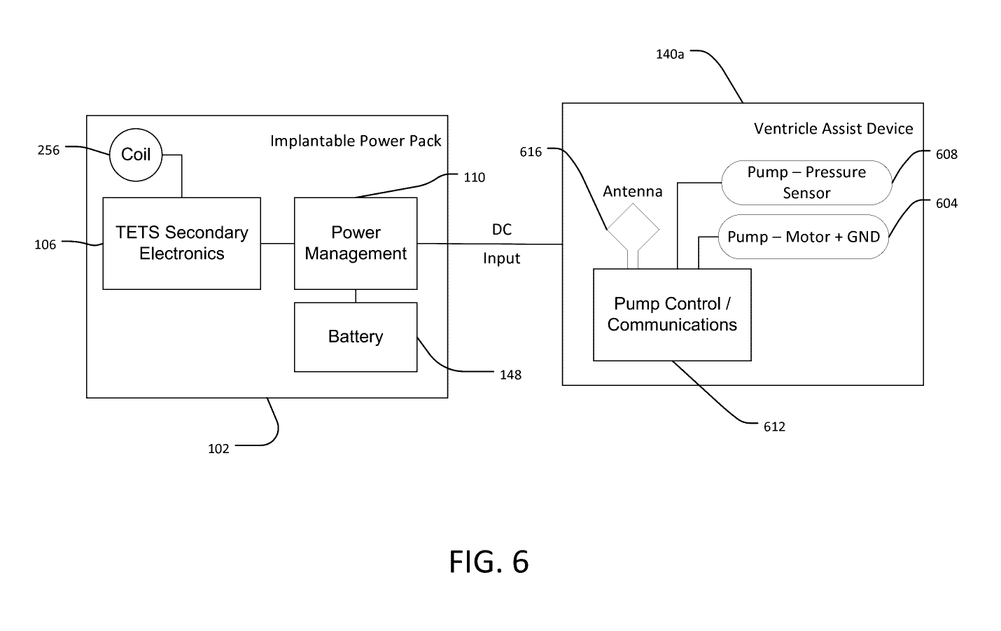

The TETS secondary electronics component 106 may connect to a power management component 110 that regulates power supplied by the rectifier 152. The power management component 110 provides electric power, via a pair of conductors 240, 244, to the various medical devices that are implanted within the subject and that receive power from the implantable power pack 102. As shown by way of example in FIG. 6, the implantable power pack 102 may provide electric power to a controller 612 that controls the operation of a heart pump 604. The power conductors 240, 244 may also supply electric power to the heart pump 604 through the controller 612. The power management component 110 may include a regulator 156, which may be implemented as a shunt type regulator that repeatedly charges and discharges a power supply capacitor. In other implementations, other types of regulators, such as a series regulator, may be used. In one embodiment, the power supply capacitor is a component of a charging circuit. The voltage across the power capacitor is output via the lines 240, 244 to the controller 612 and to the implanted medical device such as a heart pump 604.

During operation, the motor controller 612 drives the heart pump 604 to pump blood through the artificial heart assembly, drawing electric current from the power supply capacitor associated with the charging circuit. As current is drawn from the capacitor, the voltage across the capacitor decreases. To replenish the voltage on the capacitor, the power management component 110 periodically operates in a power supply mode in which electric current generated by the rectifier 152 is provided to the capacitor via the lines 240, 244. When not operating in the power supply mode, the power management component 110 operates in an idle mode in which current is not supplied to the capacitor.

In the case of shunt type regulator 156 shorting of the resonant secondary 128 may be accomplished by one or more shorting switches 272 that operate to shift the power management component 110 between the power supply mode and the idle mode. In the power supply mode, the shorting switches 272 open to allow current to flow from the internal resonant network 128, through the rectifier 152, and out to the conductor line 240/244. In idle mode, the shorting switches 272 close to short internal resonant network 128 so that current flows only within resonant network 228 rather than out to the conductor lines 240/244.

The magnitude of the output voltage across the power supply capacitor associated with regulator circuit 156 may control whether the shorting switches 272 are open or closed and thus whether the power management component 110 operates in the power supply mode or in the idle mode. For example, if the output voltage falls below a certain value, the shorting switches 272 open and the power management component 110 operates in the power supply mode. When the output voltage rises to a certain value, the shorting switches 272 close and the power supply circuit 132 operates in the idle mode. By selectively supplying current to the power supply capacitor only during certain times (i.e. the power supply mode), the voltage across the capacitor is regulated, or maintained within a predetermined voltage range, such as between about 13 and about 14 volts, for example.

In one embodiment, the shorting switches 272 are implemented as a pair of switching transistors, such as field-effect transistors, though any suitable structure may be used. For example, the shorting switches 272 may be implemented using bipolar junction transistors, and so on. The switching transistors may be configured to short diodes associated with the rectifier 152 in a conductive state and to not do so in a non-conductive state. A switching control circuit may control the conductive state of the switching transistors based on the output voltage across the power supply capacitor associated with the regulator circuit 156. When the output voltage is above a certain value, the control circuit turns on the switching transistors to short diodes associated with the rectifier 152. Here, current flows through the internal resonant network 128 and through the conductive transistors. When the output voltage is below a certain value, the control circuit turns off the switching transistors so that the diodes associated with the rectifier 152 are not shorted. Here, current is allowed to flow from the internal resonant network 128, through the rectifier 152, and out to the conductor line 240/244.

The external assembly 104 may be responsive to the internal assembly shifting between the power supply mode and the idle mode. As mentioned above, the external assembly includes a controller 124 that may be used to control the operation of the inverter 116 based on one or more characteristics of the current sensed by the sensor 220. In this regard, the controller 124 may change the frequency at which the inverter 116 operates to conserve electric power during the idle mode. During the idle mode, when electric current is not being supplied to the capacitor associated with the charging circuit, the power transmitted to the internal coil 256 by the external coil 248 is reduced in order to conserve the power of the power supply 112. This is accomplished by changing the frequency at which the inverter 116 operates.

As noted above, the internal and external coils 248, 256 may be tuned to a power transmission frequency, such as about 200 kHz. Consequently, when it is desired to transmit power to the internal coil 256, the inverter 116 is operated at the power transmission frequency to which it is tuned. However, when it is not necessary to transmit a significant amount of power, such as during the idle mode above, the frequency of the inverter 116 is changed. The frequency at which the inverter 116 operates during the power-supply mode may be changed to an odd sub-harmonic of that frequency during the idle mode. For example, the idle mode frequency may be ⅓, ⅕, 1/7, 1/9 of the power supply mode frequency. The amount of power transmitted to the internal coil 256 varies with the idle mode frequency, with less power being transmitted at the seventh subharmonic (i.e. 1/7 of the power supply mode frequency, or 28.6 kHz if the power transmission frequency is 200 kHz) than at the third subharmonic (i.e. ⅓ of the power supply mode frequency). Since odd subharmonics of a fundamental frequency still contain, in accordance with Fourier analysis, some components of the fundamental frequency, using an odd subharmonic of the power supply mode frequency during idle mode will still result in some power being transmitted to the internal coil 256, which is generally desirable.

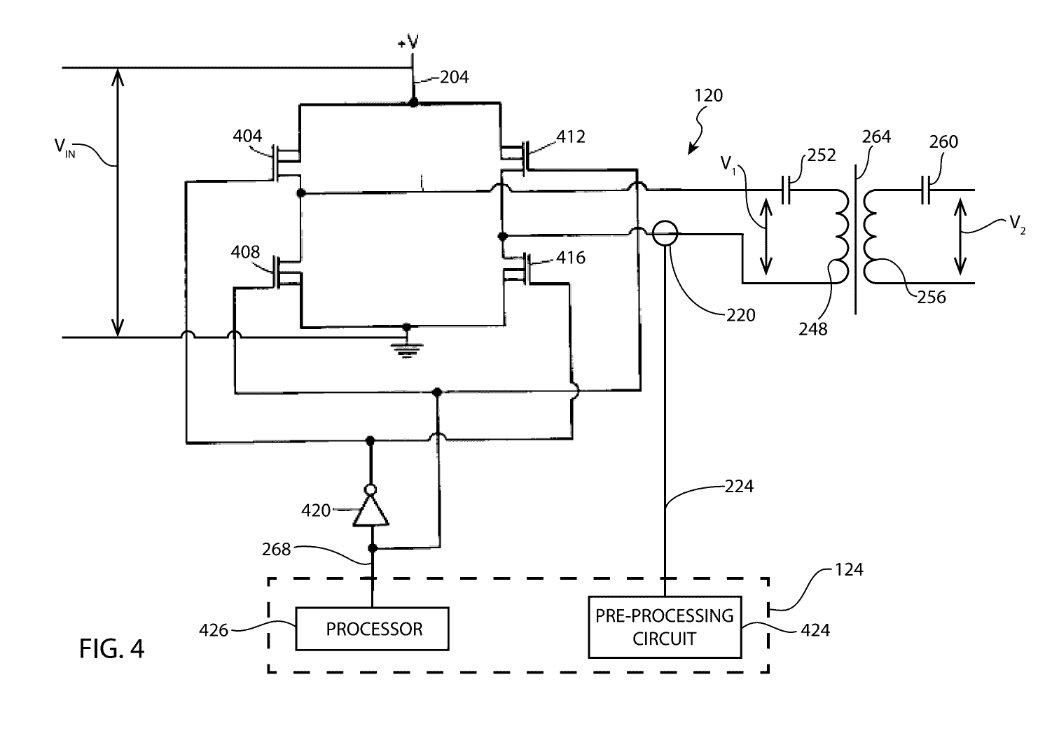

FIG. 4 is a circuit diagram that shows one implementation of the inverter 116. As shown in FIG. 4, the inverter 116 may comprise four transistors 404, 408, 412, 416, which may be metal oxide field-effect transistors (MOSFETs), connected in an H-bridge configuration. The four transistors 404, 408, 412, 416 may drive the external coil network 120 through the conductor 212. Each of the transistors 404, 408, 412, 416, may be controlled by a respective high-frequency drive signal provided on the conductor 268, with two of the drive signals being 180° out of phase, or complemented, with respect to the other two via an inverter 420. The drive signals may be 50% duty cycle square waves provided at a frequency of about 200 kHz, for example. Although a particular type of DC-to-AC converter has been described above, any type of electronic switching network that generates a high-frequency voltage may be used. For example, as an alternative to the H-bridge configuration, the inverter 116 may have transistors arranged in a voltage source half bridge configuration or in a current source configuration or in a class-DE amplifier voltage source configuration.

The inverter 116 may be connected to the controller 124 to control the operation of the inverter 116 based on one or more characteristics of the current sensed by the sensor 220. Referring to FIG. 2, the inverter 116 may be connected to the controller 124 through the conductor 268. The controller 124, in turn, may be connected to the current sensor 220 via the line 224. Referring to FIG. 4, controller 124 may include certain pre-processing circuits 424 that operate on the current signal and a processor 426 that receives input generated by the pre-processing circuit 424 based the current signal. The pre-processing circuits 424 may include circuits that accomplish such functions as current to voltage conversion, decoupling detection, interference detection, and shorting/un-shorting detection, and so on.

In one embodiment, the pre-processing circuit 424 may be configured to generate a voltage that is indicative of the magnitude of the electric current flowing through the external coil 248, where the current flowing through the external coil 248 is proportional to the voltage across the internal coil 256. During the idle mode, the shorting switches 272 are closed, which causes the voltage across the internal coil network 128 to significantly decrease. That voltage decrease causes the current in the external coil 248 to be significantly decreased, in accordance with Equation (1). Consequently, the voltage generated by the pre-processing circuit 424 decreases significantly when the power management component 110 is in the idle mode.

The output of the controller 124 may be configured to drive the inverter 116 at different frequencies depending on the voltage received from the pre-processing circuit 424. In one embodiment, the controller 124 output may be provided by the processor 426, which provides output responsive to input from the pre-processing circuit 424. When the pre-processing circuit 424 generates a voltage that is not decreased indicating that the power management component 110 is in power supply mode, the output of the controller 124 may drive the inverter 116 at a first frequency, such as 200 kHz. When the pre-processing circuit 424 generates a voltage that is decreased indicating that the power management component 110 is in idle mode, the output of the controller 124 may drive the inverter 116 at a second frequency that is an odd sub-harmonic of the frequency generated during the power supply mode.

### Power Output from the Implantable Power Pack

An implantable power pack 102 may include one or more outputs that provide a connection to one or more medical devices that are implanted within the subject. The power outputs may take the form a socket or other connector disposed on the external packaging of the implanted power pack 102. The implantable power pack 102 may connect to an implanted medical device through a wire or other conductor that attaches to the connector portion of the power pack 102. The connector may be sealed off from the remainder of the power pack 102 so that interior of power pack 102 remains sterile or other otherwise isolated from bodily fluids or other biological material in the implant environment.

FIG. 6 is a schematic illustration of an example configuration where the implantable power pack 102 provides power to a single implanted medical device. By way of example. FIG. 6 shows a configuration in which the implantable power pack provides power to a ventricle assist device 140a. The ventricle assist device 140a may generally include a pump 604 that supports the heart function of the subject by moving blood through a heart chamber or other portions of the circulatory system. A sensor 608 may detect blood pressure or other parameters within the subject's circulatory system and provide these detected measurements as feedback to the pump 604 or other system components. The pump 604 and the sensor 608 may each connect to a pump control and communications module 612 or other component that is generally configured to control the operation of the ventricle assist device 140a. In one respect, the pump control and communications module 612 may control the operation of the pump 604 and based on feedback data provided by the sensor 608. The pump control and communications module 612 may also communicate status information and/or receive commands form other devices. In this regard, the pump control and communications module 612 may include an antenna 616 or other component that provides for wireless communication.

When a power pack in accordance with this disclosure is used to provide electrical power to an implanted medical device, that medical device may be designed without its own dedicated battery or other source of electrical power. For example, a ventricle assist device is typically designed with an integrated battery or other power source that provides electrical power to ventricle assist device components such as a controller and a heart pump. Using the disclosed implantable power pack 102 as shown in FIG. 6, a ventricle assist device 140a may be designed that receives power through an external connection rather than from the typical integrated battery arrangement. Specifically, the ventricle assist device 140a may receive power in the form of a direct current input from the implanted power pack 102 from across a wire or other conductor. A ventricle assist device 140a supports the subject's heart function and thus is typically required to be in continuous operation. In this implementation, the implanted power pack 102 is typically arranged to receive a continuous power transfer from the external assembly 104 and to provide a continuous transfer of at least of a portion of that power to the ventricle assist device 140a. If power transfer from the external assembly 104 is interrupted or otherwise unavailable, the implanted power pack 102 may transfer power to the ventricle assist device 140a using the battery 148.

Ventricle assist devices or other implantable medical devices typically operate in close proximity to the organ whose function they support and are thus typically implanted at deeper locations within the body of the patient. Many conventional ventricle assist devices include an integrated battery or other power source that typically wears out and needs to be replaced at some point during the lifetime of the device. Because of their deep implant location, accessing these devices to replace batteries can thus involve very invasive surgical procedures. A ventricle assist device 140a, such as shown in FIG. 6, that omits the typical integrated battery and is designed for use with the disclosed implanted power pack 102 does not need to be accessed for these types of repairs and thus avoids the need for invasive surgery in some instances. While the battery 148 in the implantable power pack 102 may need to be replaced, the surgery needed for that replacement is less invasive because the implantable power pack 102 is located near the surface of the patient's body.

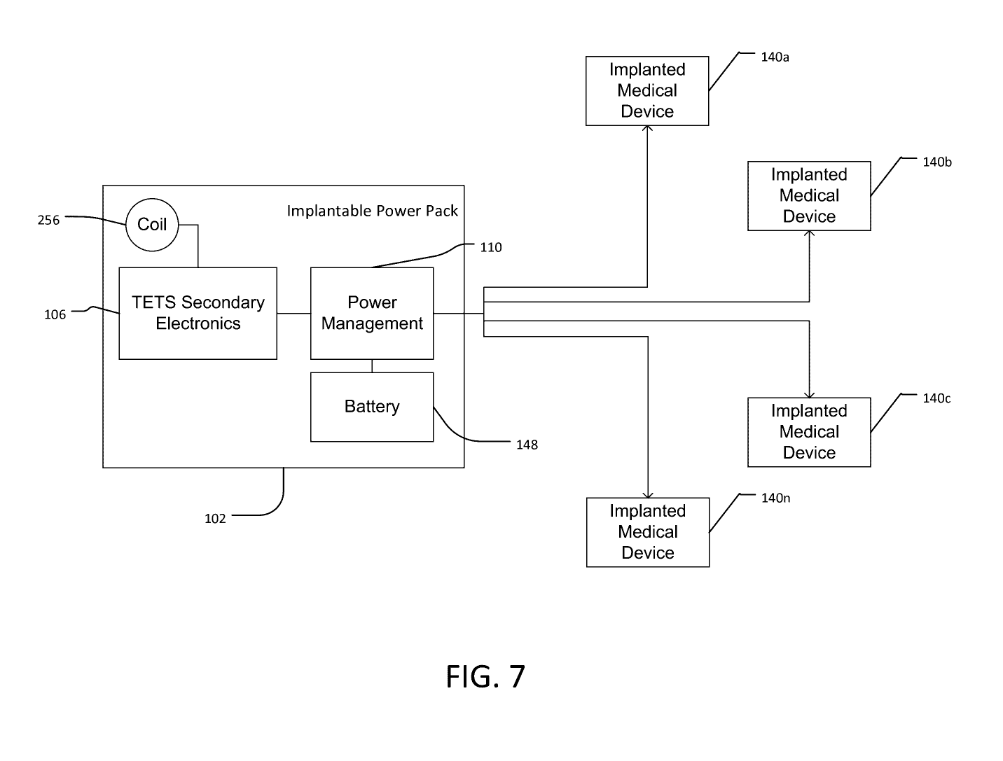

An implantable power pack 102 may have a plurality of power outputs through which the power pack 102 may connect to one or more medical devices that are implanted within the subject. FIG. 7 is a schematic illustration of an example configuration where the implantable power pack 102 provides power to multiple implanted medical devices 140a-n, which may include, for example a heart pump, an artificial heart, a right ventricle assist device, a left ventricle assist device, a BIVAD, a minimally invasive circulatory support system, a cardiac pace maker, and so on. In this configuration, the implantable power pack 102 may function as a centralized power source for the plurality of implanted medical devices 140a-n. Specifically, the various implanted medical devices 140a-n may receive power in the form of a direct current input from the implanted power pack 102 from across wires or other conductors. Here, the various implanted medical devices 140a-n may omit the integrated battery which would otherwise he used to provide electrical power. Because the various implanted medical devices 140a-n do not have separate batteries, invasive surgeries that would otherwise he needed to replace worn batteries do not need to he performed. While the battery 148 associated with the implanted power pack may need to be replaced, this may be done through a less invasive surgery because the power pack is located near the surface of the patient's body.

### Alternative Configurations and Embodiments

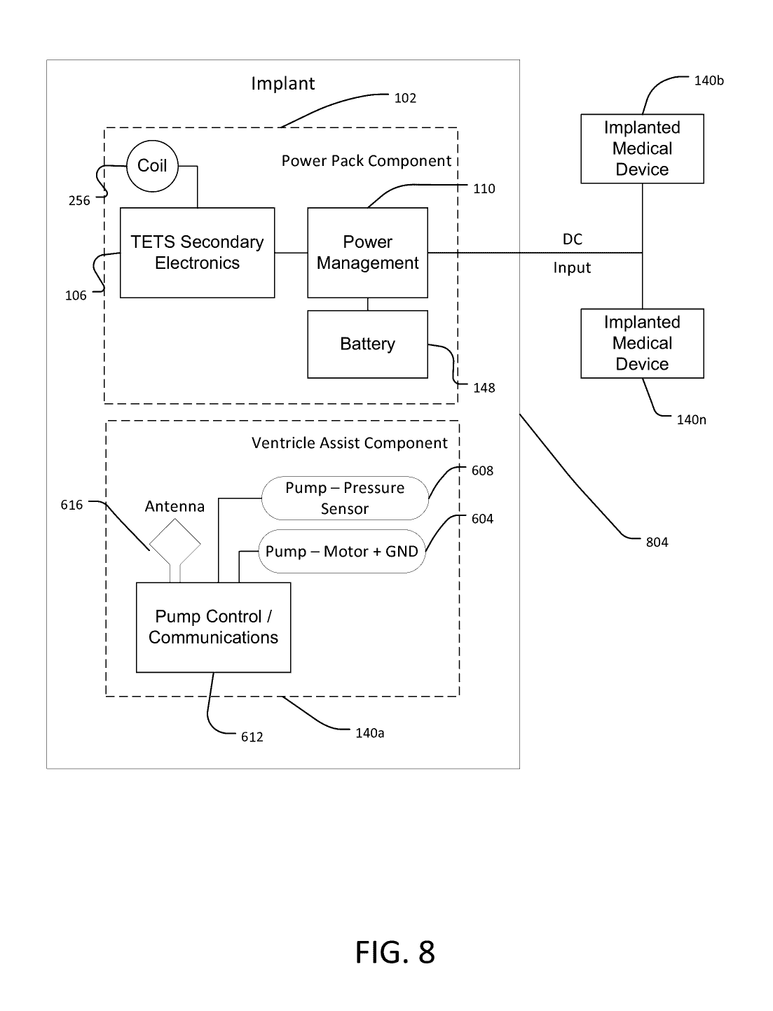

An implantable power pack in accordance with the present disclosure may be implemented in various alternative configurations. For example, as shown in FIG. 8, a single implant 804 may combine in one package an implantable power pack 102 and a ventricle assist device 140a or components thereof. The implant 804 may include power output ports that provide power to other implanted medical devices 140b-n. An implantable power pack in accordance with the present disclosure may also include a set of power packs to provide redundancy. Power pack embodiments may include a DC step-up for higher DC output voltage. In additional to what is described herein, other coil configurations could be used including the use of higher frequencies. Additional circuit and device topologies may be used in accordance with various power pack embodiments. For example, some embodiments omit a coil cable. Some embodiments include a coil and all electronics in a single package. Some embodiments have cables and all electronics inside a pump or other portion of an implanted medical device. Some embodiments include redundant coils. Some embodiments omit an external coil cable. Some embodiments include an external primary having a TETS coil and a battery integrated in one package. Some embodiments include a distributed primary battery. Using an implanted power pack in accordance with the present disclosure, alternate implant coil locations are possible such as in the beltline, primary necklace, and so on.

An implantable power pack in accordance with the present disclosure has many advantages. As described above, implanted batteries can be replaced without having to replace or surgically access controllers or other components associated with implanted medical device. More generally, controllers can be decoupled from the power management components of the system. Improved coexistence with other implanted devices may be achieved as the TETS electrical radiators are all in a single package. Stated another way, a single implantable power pack can provide power to multiple devices. Present embodiments allows the battery power to be distributed resulting in smaller implantables. Present embodiments also allow for the addition of other power systems such as a super capacitor,

The above specification, examples and data provide a complete description of the structure and use of exemplary embodiments of the invention as defined in the claims. Although various embodiments of the claimed invention have been described above with a certain degree of particularity, or with reference to one or more individual embodiments, those skilled in the art could make numerous alterations to the disclosed embodiments without departing from the spirit or scope of the claimed invention. Other embodiments are therefore contemplated. It is intended that all matter contained in the above description and shown in the accompanying drawings shall be interpreted as illustrative only of particular embodiments and not limiting. Changes in detail or structure may be made without departing from the basic elements of the invention as defined in the following claims.

The foregoing description has broad application. The discussion of any embodiment is meant only to be explanatory and is not intended to suggest that the scope of the disclosure, including the claims, is limited to these examples. In other words, while illustrative embodiments of the disclosure have been described in detail herein, the inventive concepts may be otherwise variously embodied and employed, and the appended claims are intended to be construed to include such variations, except as limited by the prior art.

The foregoing discussion has been presented for purposes of illustration and description and is not intended to limit the disclosure to the form or forms disclosed herein. For example, various features of the disclosure are grouped together in one or more aspects, embodiments, or configurations for the purpose of streamlining the disclosure. However, various features of the certain aspects, embodiments, or configurations of the disclosure may be combined in alternate aspects, embodiments, or configurations. Moreover, the following claims are hereby incorporated into this Detailed Description by this reference, with each claim standing on its own as a separate embodiment of the present disclosure.

All directional references (e,g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another. The drawings are for purposes of illustration only and the dimensions, positions, order and relative sizes reflected in the drawings attached hereto may vary.

1. An implantable power pack, comprising:
a housing;
a power output port disposed on the housing;
a resonant network included within the housing and configured to receive a wireless power transfer from an external primary power source;
a power transfer secondary electronics component included within the housing, connected to the resonant network, and configured to convert alternating current power received from the resonant network to direct current power; and
a power management component included within the housing, connected to the power transfer secondary electronics component, and configured to provide the direct current power through the power output port.

2. The implantable power pack of claim 1, further comprising a battery included within the housing and configured to be recharged by power transferred from the external primary power source.

3. The implantable power pack of claim 1, wherein the resonant network includes a coil and a capacitor.

4. The implantable power pack of claim 1, wherein the power transfer secondary electronics component includes a rectifier.

5. The implantable power pack of claim 1, wherein the power management component includes regulator.

6. The implantable power pack of claim 1, further comprising a plurality power output ports disposed on the housing wherein the power management component provides direct current power through the plurality of power output ports.

7. The implantable power pack of claim 1, wherein the power management component provides electric power to a left ventricle assist device that is connected to the output port.

8. The implantable power pack of claim 1, wherein the housing is implanted within a subject in a location that is adjacent to the skin of the subject.

9. The implantable power pack of claim 1, wherein the power management component shifts between a power supply mode and an idle mode depending on power demanded from an implanted medical device that is connected to the output port.

10. An implantable system, comprising:
a power pack configured to receive a wireless power transfer from an external primary power source and to provide power through a power output port disposed on a housing of the power pack; and
a medical device configured to connect to the power output port of the power pack and to receive power from the power pack through a power input port disposed on a housing of the medical device, wherein the housing of medical device does not have a battery included therein.

11. The implantable system of claim 10, wherein the power pack comprises:
a resonant network included within the housing and configured to receive a wireless power transfer from an external primary power source;
a power transfer secondary electronics component included within the housing, connected to the resonant network, and configured to convert alternating current power received from the resonant network to direct current power;
a power management component included within the housing, connected to the power transfer secondary electronics component, and configured to provide the direct current power through the power output port; and
a battery included within the housing and configured to be recharged by power transferred from the external primary power source.

12. The implantable system of claim 10, wherein the medical device is a ventricle assist device comprising:
a pump that supports a heart function of a subject within whom the housing is implanted;
a controller configured to control the operation of the pump;
a sensor configured to detect pressure within a circulatory system of the subject and to provide the detected pressure as feedback to the controller; and
an antenna that provides for wireless communication to external components;
wherein the controller is configured to communicate status information and/or receive commands through the antenna.

13. An implantable medical device, comprising:
a housing;
a power output port disposed on the housing;
a ventricle assist component included within the housing;
a power pack component included within the housing and connected to the ventricle assist section and to the power output port, the power pack section configured to receive a wireless power transfer from an external primary power source and to provide power to the ventricle assist section and to an additional implanted medical device through the power output port.

14. The implantable medical device of claim 13, wherein the ventricle assist component comprises:
a pump that supports a heart function of a subject within whom the housing is implanted;
a controller configured to control the operation of the pump;
a sensor configured to detect pressure within a circulatory system of the subject and to provide the detected pressure as feedback to the controller; and
an antenna that provides for wireless communication to external components;
wherein the controller is configured to communicate status information and/or receive commands through the antenna.

15. The implantable medical device of claim 13, wherein the power pack component comprises:
a resonant network included within the housing and configured to receive a wireless power transfer from an external primary power source;
a power transfer secondary electronics component included within the housing, connected to the resonant network, and configured to convert alternating current power received from the resonant network to direct current power;
a power management component included within the housing, connected to the power transfer secondary electronics component, and configured to provide the direct current power through the power output port; and
a battery included within the housing and configured to be recharged by power transferred from the external primary power source.

16. A ventricle assist device, comprising:
a housing;
a power input port disposed on the housing;
a pump that supports a heart function of a subject within whom the housing is implanted; and
a controller configured to control the operation of the pump;
wherein the pump and the controller receive electric power through the power input port and the housing does not have a battery included therein.

17. The implantable medical device of claim 16, further comprising
a sensor configured to detect pressure within a circulatory system of the subject and to provide the detected pressure as feedback to the controller.

18. The implantable medical device of claim 17, further comprising
an antenna that provides for wireless communication to external components;
wherein the controller is configured to communicate status information and/or receive commands through the antenna.
